(19) United States
(12) Patent Application Publication
(10) Pub. No.: US 2002/0193404 A1
(43) Pub. Date: Dec. 19, 2002

(54) Use of bicyclic heterocycles for the treatment and prevention of arterial thrombotic disease

(76) Inventors: Norbert Hauel, Schemmerhofen (DE); Wolfgang Wienen, Biberach/Rissegg (DE); Jean-Marie Stassen, Lubbeek (BE)

Correspondence Address: BOEHRINGER INGELHEIM CORPORATION, 900 RIDGEBURY ROAD, P. O. BOX 368, RIDGEFIELD, CT, 06877, US

(21) Appl. No.: 10/137,895

(22) Filed: May 2, 2002

Related U.S. Application Data
(60) Provisional application No. 60/301,899, filed on Jun. 28, 2001.

(30) Foreign Application Priority Data
May 25, 2001 (DE) DE 101 25 478.4

(51) Int. Cl. A61K 31/4709

(52) U.S. Cl. 514/314

(57) ABSTRACT

The present invention relates to a method of treating and preventing arterial thrombotic diseases, comprising administering an effective amount of one of the compounds

A=1-methyl-2-[(4-amidinophenyl)-oxymethyl]-5-[N-(hydroxycarbonylmethyl)-quinoline-8-sulphonylamino]-benzimidazole and

B=1-methyl-2-[N-(4-amidinophenyl)-aminomethyl]-5-[N-(hydroxycarbonylmethyl)-quinoline-8-sulphonylamino]-benzimidazole,

the physiologically acceptable salts thereof or the mixtures thereof, and the use of these compounds for preparing corresponding pharmaceutical compositions.

Benefit of U.S. Provisional Application Serial No. 60/301,899, filed on Jun. 28, 2001 is hereby claimed.

### BACKGROUND OF THE INVENTION

1. Technical Field

The invention relates to a method for treating or preventing arterial thrombotic disease by means of the administration of 1-methyl-2-[(4-amidinophenyl)-oxymethyl]-5-[N-(hydroxycarbonylmethyl)-quinoline-8-sulphonylamino]-benzimidazole or 1-methyl-2-[N-(4-amidinophenyl)-aminomethyl]-5-[N-(hydroxycarbonylmethyl)-quinoline-8-sulphonylamino]-benzimidazole.

2. Background Information

WO 00/08014 describes bicyclic heterocycles which inhibit thrombin, the central enzyme of the blood clotting cascade, and are thus particularly suitable for the treatment and prophylaxis of thromboses in venous blood vessels (D. P. Thomas: Pathogenesis of Venous Thrombosis, in: Haemostasis and Thrombosis (ed. Bloom, Forbes, Thomas and Tuddenham), Churchill Livingstone, New York, 3^{rd} Edition, 1994, page 1335-1347). The formation of thrombi in the arterial vascular bed is, however, only slightly suppressed by the inhibition of thrombin, as the formation of a thrombus is chiefly triggered by the adhesion and aggregation of blood platelets (M. A. Packham, R. L. Kinlough-Rathbone: Mechanisms of Atherogenesis and Thrombosis, in: Haemostasis and Thrombosis (ed. Bloom, Forbes, Thomas and Tuddenham), Churchill Livingstone, New York, 3^{rd} Edition, 1994, page 1107-1138). Selective inhibitors of blood clotting (such as e.g. coumarin derivatives) therefore serve predominantly to prevent venous occlusion and have no utility in the acute treatment of arterial occlusive diseases. On the other hand, inhibitors of platelet aggregation, especially parenteral fibrinogen receptor antagonists (e.g. Abciximab (Rheopro)) are used to prevent any new formation of platelet-rich thrombi after recanalisation of the constricted blood vessels.

### BRIEF SUMMARY OF THE INVENTION

Surprisingly, it has now been found that two of the substances described in WO 00/08014, namely

A=1-methyl-2-[(4-amidinophenyl)-oxymethyl]-5-[N-(hydroxycarbonylmethyl)-quinoline-8-sulphonylamino]-benzimidazole

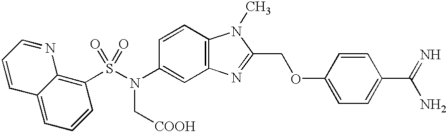

and

B=1-methyl-2-[N-(4-amidinophenyl)-aminomethyl]-5-[N-(hydroxycarbonylmethyl)-quinoline-8-sulphonylamino]-benzimidazole

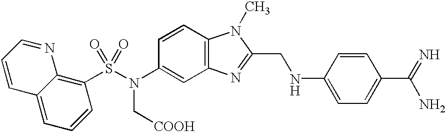

in addition to inhibiting clotting also have a very powerful effect on the human fibrinogen receptor (glycoprotein IIb/IIIa). This surprisingly advantageous combination of pharmacological properties, namely the suppression of fibrin formation by inhibiting thrombin, on the one hand, and the prevention of blood platelet aggregation by blocking the fibrinogen receptor, on the other hand, means that these active substances are particularly suitable for treating arterial thrombosis.

Thus, the invention resides in the novel recognition that arterial thrombotic disease can be treated or prevented by means of the administration these two known compounds. The invention thus provides a method for treating or preventing arterial thrombotic disease which comprises the administration of an antithrombotic or prophylatic amount of either of either 1-methyl-2-[(4-amidinophenyl)-oxymethyl]-5-[N-(hydroxycarbonylmethyl)-quinoline-8-sulphonylamino]-benzimidazole or 1-methyl-2-[N-(4-amidinophenyl)-aminomethyl]-5-[N-(hydroxycarbonylmethyl)-quinoline-8-sulphonylamino]-benzimidazole.

### DETAILED DESCRIPTION OF THE INVENTION

With regard to the preparation of compounds A and B reference is made expressly to WO 00/08014.

The inhibiting effect of the two substances on blood clotting was measured by determining the prolongation of aPTT (activated partial thromboplastin time) as follows:

Materials:

Plasma, from citrated human blood

PTT reagent, Boehringer Mannheim (524298)

Calcium solution (0.025 mol/L), Behring Werke, Marburg (ORH 056/57)

Diethyl barbiturate acetate buffer, Behring Werke, Marburg (ORWH 60/61)

Procedure:

The aPTT was determined using a Biomatic—B 10 coagulometer made by Messrs Desaga (Wiesloch). The test substance was placed in the test tubes prescribed by the manufacturer with 0.1 ml of human citrated plasma and 0.1 ml of PTT reagent. The preparations were incubated for three minutes at 37° C. The clotting reaction was started by the addition of 0.1 ml of calcium solution. The time taken for the plasma to clot was measured by the apparatus as the calcium solution was added. Analogous test preparations to which 0.1 ml of diethyl barbiturate acetate buffer had been added were used as controls. According to the definition of EC₂₀₀, the substance concentration at which the aPTT time was double that of the control was determined using a dosage-activity curve.

The inhibiting effect on the human fibrinogen receptor (glycoprotein IIb/IIIa) was investigated as follows:

Inhibition of Binding of ³H-BIBU 52 to Human Thrombocytes:

A suspension of human thrombocytes was incubated with ³H-BIBU 52 [=(3S,5S)-5-[(4′-amidino-4-biphenylyl)oxymethyl]-3-[(carboxy)methyl]-2-pyrrolidinone[3-³H-4-biphenylyl]], which replaces the ligand ¹²⁵I-fibrinogen known from the literature (cf. DE-A-4 214 245), and various concentrations of the substance being tested. The free and bound ligands were separated by centrifugation and quantitatively determined by scintillation counting. The inhibition of the ³H-BIBU 52 binding by the test substance was determined from the measurements obtained. To do this, donor blood was taken from an anticubital vein and anticoagulated with trisodium citrate (final concentration: 13 mM). The blood was centrifuged for ten minutes at 170× g and the supernatant platelet-rich plasma (PRP) was removed. The remaining blood was sharply centrifuged once more to obtain plasma. The PRP was diluted 1:10 with autologous plasma. 750 μl thereof were incubated with 50 μl of physiological saline solution, 100 μl of test substance solution, 50 μl of ¹⁴C-sucrose (3,700 Bq) and 50 μl of ³H-BIBU 52 (final concentration: 5 nM) at ambient temperature for 20 minutes. In order to measure the non-specific binding, 5 μl of BIBU 52 (final concentration: 30 μM) were used instead of the test substance. The samples were centrifuged for 20 seconds at 10000× g and the supernatant was removed. 100 μl thereof were assayed in order to quantify the free ligand. The pellet was dissolved in 500 μl of 0.2N NaOH, 450 μl were mixed with 2 ml of scintillator and 25 μl of 5N HCl and assayed. The residual plasma still remaining in the pellet was determined from the ¹⁴C content while the bound ligand was determined from the ³H measurement. After subtraction of the non-specific binding the pellet activity was plotted against the concentration of test substance and the concentration which produced a 50% inhibition of binding (IC₅₀) was determined.

The following Table contains the values found:

| | aPTT | inhibition of fibrinogen |
|---|---|---|
| | (EC₂₀₀ in | receptor |
| Substance | μMol/L) | (IC₅₀ in μMol/L) |
| A | 0.013 | 0.006 |
| B | 0.026 | 0.049 |

In view of their pharmacological properties these two active substances A and B and the physiologically acceptable salts thereof are particularly suitable for preventing and treating arterial thrombotic diseases, such as for example preventing coronary thrombosis, preventing the occlusion of shunts or stents, preventing reocclusions after bypass operations or angioplasty and for antithrombotic support (preventing the reocclusion of coronary arteries) during and after thrombolytic treatment, such as e.g.

with rt-PA or streptokinase.

The present invention thus relates to a method of treating and preventing arterial thrombotic diseases, particularly the abovementioned arterial thrombotic diseases, comprising administering an effective amount of one of the compounds

A=1-methyl-2-[(4-amidinophenyl)-oxymethyl]-5-[N-(hydroxycarbonylmethyl)-quinoline-8-sulphonylamino]-benzimidazole and

B=1-methyl-2-[N-(4-amidinophenyl)-aminomethyl]-5-[N-(hydroxycarbonylmethyl)-quinoline-8-sulphonylamino]-benzimidazole, the physiologically acceptable salts thereof or the mixtures thereof.

The dosage required to obtain such an effect is conveniently 0.001 to 1.0 mg/kg, preferably 0.003 to 0.3 mg/kg when administered by intravenous bolus; and 0.001 to 0.5 mg/kg/h, preferably 0.003 to 0.1 mg/kg/h by intravenous infusion.

The invention further relates to the use of one of the abovementioned compounds A and B, the physiologically acceptable salts thereof or the mixtures thereof for preparing a pharmaceutical composition for the treatment and prevention of the arterial thrombotic diseases mentioned hereinbefore.

What is claimed is:

1. A method of treating or preventing arterial thrombotic disease, comprising administering an effective amount of a compound selected from the group consisting of
(a) 1-methyl-2-[(4-amidinophenyl)-oxymethyl]-5-[N-(hydroxycarbonylmethyl)-quinoline-8-sulphonylamino]-benzimidazole; and
(b) 1-methyl-2-[N-(4-amidinophenyl)-aminomethyl]-5-[N-(hydroxycarbonylmethyl)-quinoline-8-sulphonylamino]-benzimidazole,
or a physiologically acceptable salt thereof.

2. The method according to claim 1 for the prevention of coronary thrombosis.

3. The method according to claim 1 for preventing the occlusion of shunts or stents.

4. The method according to claim 1 for preventing reocclusions after bypass operations or angioplasty.

5. The method according to claim 1 for antithrombotic support and prevention of reocclusions of coronary arteries during and after thrombolytic treatment.

6. The method according to any one of claims 1, 2, 3, 4 or 5, wherein the compound is administered as an intravenous bolus or by intravenous infusion.

7. The method according to claim 6, wherein the compound is administered as an intravenous bolus at a dosage of 0.001 to 1.0 mg/kg or by intravenous infusion at a dosage of 0.001 to 0.5 mg/kg/h.
